(19) United States
(12) Patent Application Publication
(10) Pub. No.: US 2019/0247373 A1
(43) Pub. Date: Aug. 15, 2019

(54) METHODS FOR TREATING FIBROSIS

(71) Applicant: CHILDREN'S HOSPITAL MEDICAL CENTER, Cincinnati, OH (US)

(72) Inventors: Anil Goud JEGGA, West Chester, OH (US); Satish Kumar MADALA, Blue Ash, OH (US)

(73) Assignee: CHILDREN'S HOSPITAL MEDICAL CENTER, Cincinnati, OH (US)

(21) Appl. No.: 16/317,293

(22) PCT Filed: Jul. 13, 2017
(86) PCT No.: PCT/US2017/041898

§ 371 Date: Jan. 11, 2019

Related U.S. Application Data
(60) Provisional application No. 62/362,169, filed on Jul. 14, 2016.

(51) Int. Cl. A61K 31/439; A61K 31/4412; A61K 31/405; A61P 11/00

(52) U.S. Cl. CPC A61K 31/439; A61P 11/00; A61K 31/405; A61K 31/4412

(57) ABSTRACT

Some embodiments of the invention include methods for treating an animal for fibrosis comprising one or more administrations of one or more compositions comprising one or more opioid receptor inhibitors. Other embodiments of the invention further include other fibrosis treatments. Still other embodiments of the invention include methods for treating a human for idiopathic pulmonary fibrosis, comprising administering one or more compositions comprising naltrexone and optionally administering one or more compositions comprising pirfenidone, nintedanib, or both. Additional embodiments of the invention are also discussed herein.

### CROSS-REFERENCE TO RELATED APPLICATIONS

This application claims the benefit of U.S. Provisional Application No. 62/362,169, filed Jul. 14, 2016 which is herein incorporated by reference in its entirety.

### BACKGROUND

Fibrosis is the formation of excess fibrous connective tissue. In some instances, fibrosis results in accumulation of extracellular matrix proteins.

Several compounds are known to treat fibrosis, but do so inadequately. For example, pirfenidone and nintedanib are newly FDA-approved drugs for the treatment of idiopathic pulmonary fibrosis. However, pirfenidone showed no effect on respiratory symptoms. And neither pirfenidone nor nintedanib had any effect on mortality. Thus, attempts to develop a clinically effective fibrosis have been unsuccessful, and there is still a need to find treatments for fibrosis.

Certain embodiments of the invention address one or more of the deficiencies described above. For example, some embodiments of the invention include methods for treating an animal for fibrosis comprising one or more administrations of one or more compositions comprising one or more opioid receptor inhibitors. Other embodiments of the invention further include other fibrosis treatments. Still other embodiments of the invention include methods for treating a human for idiopathic pulmonary fibrosis, comprising administering one or more compositions comprising naltrexone and optionally administering one or more compositions comprising pirfenidone, nintedanib, or both. Additional embodiments of the invention are also discussed herein.

### SUMMARY

Some embodiments of the invention include a method for treating an animal for fibrosis, comprising one or more administrations of one or more compositions comprising one or more opioid receptor inhibitors, wherein the compositions may be the same or different if there is more than one administration. In other embodiments, one or more opioid receptor inhibitors inhibits one or more of a delta opioid receptor, deltal opioid receptor, delta2 opioid receptor, a kappa opioid receptor, kappa1 opioid receptor, kappa2 opioid receptor, kappa3 opioid receptor, a mu opioid receptor, mu1 opioid receptor, mu2 opioid receptor, mu3 opioid receptor, a nociceptin opioid receptor, a zeta opioid receptor, a sigma opioid receptor, or an epsilon opioid receptor. In yet other embodiments, one or more opioid receptor inhibitors is a mu opioid receptor (MOR) antagonist, an MOR partial antagonist, an MOR inverse agonist, an MOR partial inverse agonist, a kappa opioid receptor (KOR) antagonist, a KOR partial antagonist, a KOR inverse agonist, a KOR partial inverse agonist, a delta opioid receptor (DOR) antagonist, a DOR partial antagonist, a DOR inverse agonist, a DOR partial inverse agonist, a nociceptin opioid receptor inhibitor, a zeta opioid receptor inhibitor, a sigma opioid receptor inhibitor, a epsilon opioid receptor inhibitor, or a combination thereof. In still other embodiments, one or more opioid receptor inhibitors is an MOR antagonist, an MOR partial antagonist, an MOR inverse agonist, an MOR partial inverse agonist, a KOR antagonist, a KOR partial antagonist, a KOR inverse agonist, a KOR partial inverse agonist, a DOR antagonist, a DOR partial antagonist, a DOR inverse agonist, a DOR partial inverse agonist, or a combination thereof. In other embodiments, one or more opioid receptor inhibitors is one or more of alvimopan, AT-076 ((3R)-7-hydroxy-N-[(2S)-1-[4-(3-hydroxyphenyl)piperidin-1-yl]-3-methylbutan-2-y]-1,2,3,4-tetrahydroisoquinoline-3-carboxamide), axelopran, bevenopran, buprenorphine, buprenorphine/samidorphan, buprenorphine/naltrexone, butorphanol, CERC-501 (4-(4-{[(2S)-2-(3,5-Dimethylphenyl)-1-pyrrolidinyl]methyl}phenoxy)-3-fluorobenzamide; CAS number 1174130-61-0), cyprodime, dezocine, diprenorphine, eptazocine, J-113,397 (1-[(3R,4R)-1-cyclooctylmethyl-3-hydroxymethyl-4-piperidyl]-3-ethyl-1, 3-dihydro-2H-benzimidazol-2-one; CAS number 256640-45-6) or a racemic mixture thereof, JDTic ((3R)-7-Hydroxy-N-[(2S)-1-[(3R,4R)-4-(3-hydroxyphenyl)-3,4-dimethylpiperidin-1-yl]-3-methylbutan-2-yl]1-1,2,3,4-tetrahydroisoquinoline-3-carboxamide; CAS number 361444-66-8), JTC-801 (N-(4-amino-2-methylquinolin-6-yl)-2-[(4-ethylphenoxy)methyl]benzamide; CAS number 244218-51-7), levallorphan, levorphanol, LY-2940094 (2-[4-[(2-chloro-4,4-difluoro-spiro[5H-thieno[2,3-c]pyran-7,4′-piperidine]-1′-yl)methyl]-3-methyl-pyrazol-1-yl]-3-pyridyl]methanol; CAS number 1307245-86-8), methylnaltrexone, methylsamidorphan, nalbuphine, naldemedine, nalmefene, nalodeine, nalorphine, nalorphine dinicotinate, naloxegol, naloxone, 6β-naltrexol, naltrexone, naltrindole, norbinaltorphimine, pentazocine, PF-4455242 (2-Methyl-N-{[2′-(1-pyrrolidinylsulfonyl)-4-biphenylyl]methyl}-1-propanamine; CAS number 1202647-54-8), phenazocine, SB-612,111 (i.e., (5S,7S)-7-{[4-(2,6-dichlorophenyl)piperidin-1-yl]methyl}-1-methyl-6,7,8,9-tetrahydro-5H-benzo[7]annulen-5-ol; CAS number 371980-98-2), samidorphan; or a salt, ester, or solvate of any of the aforementioned. In additional embodiments, one or more opioid receptor inhibitors is one or more of diprenorphine, levallorphan, nalmefene, nalorphine, nalorphine dinicotinate, naloxone, naltrexone, samidorphan; or a salt, ester, or solvate of any of the aforementioned. In some instances, one or more opioid receptor inhibitors is one or more of diprenorphine, levallorphan, nalmefene, nalorphine, nalorphine dinicotinate, naloxone, naltrexone, or samidorphan. In other instances, one or more opioid receptor inhibitors is nalmefene, naloxone, naltrexone, or samidorphan. In certain embodiments, one or more opioid receptor inhibitors is naltrexone.

In some embodiments, the amount of the one or more opioid receptor inhibitors is from about 0.0001% (by weight total composition) to about 99%. In other embodiments, at least one of the one or more compositions further comprises a formulary ingredient. In still other embodiments, at least one of the one or more compositions is a pharmaceutical composition. In certain embodiments, at least one of the one or more administrations comprises parenteral administration, a mucosal administration, intravenous administration, depot injection, subcutaneous administration, topical administration, intradermal administration, oral administration, sublingual administration, intranasal administration, or intramuscular administration. In still other embodiments, at least one of the one or more administrations comprises a depot injection or an oral administration. In yet other embodiments, if there is more than one administration at least one composition used for at least one administration is different from the composition of at least one other administration. In certain embodiments, the compound of at least one of the one or more compositions is administered to the animal in an amount of from about 0.005 mg/kg animal body weight to about 100 mg /kg animal body weight.

In some embodiments, the animal is a human, a rodent, or a primate. In other embodiments, the animal is in need of treatment of fibrosis (e.g., lung fibrosis, pulmonary fibrosis, cystic fibrosis, idiopathic pulmonary fibrosis (IPF), or radiation-induced lung injury; or lung fibrosis, skin fibrosis, kidney fibrosis, liver fibrosis, heart fibrosis, or brain fibrosis; or lung fibrosis, skin fibrosis, kidney fibrosis, heart fibrosis, or brain fibrosis; or lung fibrosis, kidney fibrosis, heart fibrosis, or brain fibrosis; or lung fibrosis, heart fibrosis, or brain fibrosis). In certain embodiments, the method is for treating lung fibrosis, skin fibrosis, kidney fibrosis, liver fibrosis, heart fibrosis, brain fibrosis, arterial stiffness, arthrofibrosis, crohn's disease, dupuytren's contracture, keloid, mediastinal fibrosis, myelofibrosis, peyronie's disease, nephrogenic systemic fibrosis, progressive massive fibrosis (e.g., a complication of coal workers' pneumoconiosis), retroperitoneal fibrosis, scleroderma/systemic sclerosis, or adhesive capsulitis. In yet other embodiments, the method is for treating lung fibrosis, pulmonary fibrosis, cystic fibrosis, idiopathic pulmonary fibrosis (IPF), radiation-induced lung injury, skin fibrosis, kidney fibrosis, liver fibrosis, cirrhosis, heart fibrosis, atrial fibrosis, endomyocardial fibrosis, or myocardial infarction. In still other embodiments, the method is for treating lung fibrosis, pulmonary fibrosis, cystic fibrosis, idiopathic pulmonary fibrosis (IPF), radiation-induced lung injury, skin fibrosis, kidney fibrosis, heart fibrosis, atrial fibrosis, endomyocardial fibrosis, or myocardial infarction. In some instances, the method is for treating lung fibrosis, kidney fibrosis, liver fibrosis, heart fibrosis, or brain fibrosis. In other instances, the method is for treating lung fibrosis, kidney fibrosis, heart fibrosis, or brain fibrosis. In some embodiments, the fibrosis is not liver fibrosis. In yet other embodiments, the fibrosis is not fibrosis related to cirrhosis.

In some embodiments, the method further comprises one or more other fibrosis treatments. In other embodiments, the method further comprises one or more other fibrosis treatments and the other fibrosis treatment comprises administering one or more of an antibiotic, an anti-inflammatory drug, a mucus thinner, or an antifibrotic medication. In additional embodiments, the method further comprises one or more other fibrosis treatments and the other fibrosis treatment comprises administering pirfenidone, nintedanib, or both. In still other embodiments, the method further comprises one or more other fibrosis treatments and the other fibrosis treatment comprises administering one or more non-drug respiratory therapies.

Some embodiments of the invention include a method for treating a human for lung fibrosis, comprising one or more administrations of one or more compositions comprising naltrexone and optionally pirfenidone, nintedanib, or both, wherein the compositions may be the same or different if there is more than one administration.

Other embodiments of the invention include a method for treating a human for IPF, comprising administering one or more compositions comprising naltrexone and optionally administering one or more compositions comprising pirfenidone, nintedanib, or both.

Other embodiments of the invention are also discussed herein.

### BRIEF DESCRIPTION OF THE DRAWINGS

The following drawings form part of the present specification and are included to further demonstrate certain aspects of the present invention. The invention may be better understood by reference to one or more of these drawings in combination with the description of specific embodiments presented herein.

FIG. 1: Naltrexone attenuates IPF-specific pathological gene networks. Comparison of IPF patient gene expression profile (GEP) with naltrexone-treated cells GEPs in the context of known ECM and fibrogenic signaling pathway genes. Panel A shows the ECM/fibrosis genes negatively correlated between IPF upregulated genes with naltrexone-downregulated genes while panel B shows the opposite.

FIG. 2: Naltrexone attenuates IPF-specific pathological gene networks involved in proliferation, migration, ECM production and fibrosis. Network representation of enriched processes in negatively correlated gene sets between IPF lungs and naltrexone treated cells from the LINCS database. Circles to the left of “”Extracellular matrix” and “Fibrosis” octagons represent genes up regulated in IPF patients. Circles to the right of “”Extracellular matrix” and “Fibrosis” octagons represent genes down regulated in IPF patients. Octagons represent some of the top enriched biological processes.

FIG. 3: H&E-stained lung biopsies from a patient with IPF (A) and from a transgenic mouse (B) that overexpressed TGFs in airway epithelial cells. TGFα mice developed fibrotic lesions in the lung subpleura and parenchyma with histological features similar to human IPF.

FIG. 4: The endogenous opioid receptor ligand, proenkephalin A (PENK) levels were elevated in pulmonary fibrosis. The transcripts of PENK were elevated in the lungs during pulmonary fibrosis in TGFα mice on Dox for 14 and 42 days compared to control mice on Dox for 0 days. One-Way ANOVA (N=4−5/group; *P<0.05).

FIG. 5: Mouse model of bleomycin-induced pulmonary fibrosis. Masson's trichrome-stained lung sections show extensive collagen deposition in mice treated intradermally with bleomycin (6 U/kg body weight; 5 days/week for 4 weeks) (B) compared to saline (A).

FIG. 6: Naltrexone therapy attenuates ECM and proliferative gene expression in pulmonary fibrosis. (A) Experimental schemata of naltrexone therapy study in vivo. Three groups of mice on Dox for two weeks were treated with either vehicle or naltrexone (10 mg/kg or 50mg/kg bodyweight, b.i.d) and sacrificed after a week of treatment while continued on Dox. Total lung RNA was analyzed for the expression of: (B) ECM genes (FN1, Col5α, and Col6α) and (C) proliferation genes (Aurka, IL-6, and Calcb). One-Way ANOVA (N=4/group; *P<0.05).

FIG. 7: Naltrexone therapy attenuates body weight loss in pulmonary fibrosis. (A) Experimental schemata of naltrexone therapy study in vivo. Three groups of mice on Dox for three weeks were treated with either vehicle or naltrexone (80 mg/kg bodyweight, b.i.d) and then sacrificed after three weeks of treatment while continued on Dox. (B) The loss of body weight was attenuated with naltrexone therapy compared to vehicle treatment in TGFα mice. One-Way ANOVA (N=4−6/group; *P<0.05).

FIG. 8: Naltrexone therapy attenuates increase in lung weights in pulmonary fibrosis. Three groups of mice on Dox for three weeks were treated with either vehicle or naltrexone (80 mg/kg bodyweight, b.i.d) and then sacrificed after three weeks of treatment while continued on Dox. The increase in lung weights was attenuated with naltrexone therapy compared to vehicle treatment in TGFα mice. One-Way ANOVA (N=4−6/group; *P<0.05).

FIG. 9: Naltrexone therapy attenuates lung function decline in pulmonary fibrosis. Three groups of mice on Dox for three weeks were treated with either vehicle or naltrexone (80 mg/kg bodyweight, b.i.d) and then sacrificed after three weeks of treatment while continued on Dox. Decrease in the lung function (A,,

Resistance; B, Compliance; C, Elastance) was attenuated with naltrexone therapy compared to vehicle treatment in TGFα mice. One-Way ANOVA (N=4−6/group; *P<0.05).

FIG. 10: Naltrexone treatment attenuates ECM and proliferation gene expression in IPF fibroblasts. Expression of ECM genes (FN1, Col3α, and αSMA) and proliferation gene (Aurka) was attenuated in IPF fibroblasts treated with naltrexone (10 μM) compared to vehicle treatment for 24 hrs in IPF fibroblasts. Unpaired t-test (N=3/group; *P<0.05).

### DETAILED DESCRIPTION

While embodiments encompassing the general inventive concepts may take diverse forms, various embodiments will be described herein, with the understanding that the present disclosure is to be considered merely exemplary, and the general inventive concepts are not intended to be limited to the disclosed embodiments.

Some embodiments of the invention include methods for treating an animal for fibrosis comprising one or more administrations of one or more compositions comprising one or more opioid receptor inhibitors. Other embodiments of the invention further include other fibrosis treatments. Still other embodiments of the invention include methods for treating a human for idiopathic pulmonary fibrosis, comprising administering one or more compositions comprising naltrexone and optionally administering one or more compositions comprising pirfenidone, nintedanib, or both. Additional embodiments of the invention are also discussed herein.

### Treatments of Disease

Some embodiments of the invention include treatment of disease (e.g., fibrosis) by administering one or more opioid receptor inhibitors. One or more opioid receptor inhibitors (e.g., naltrexone) can be administered to animals by any number of suitable administration routes or formulations. One or more opioid receptor inhibitors (e.g., naltrexone) can also be used to treat animals for a variety of diseases. Animals include but are not limited to mammals, primates, monkeys (e.g., macaque, rhesus macaque, or pig tail macaque), humans, canine, feline, bovine, porcine, avian (e.g., chicken), mice, rabbits, and rats. As used herein, the term “subject” refers to both human and animal subjects.

The route of administration of one or more opioid receptor inhibitors (e.g., naltrexone) can be of any suitable route. Administration routes can be, but are not limited to the oral route, the parenteral route, the cutaneous route, the nasal route, the rectal route, the vaginal route, and the ocular route. In other embodiments, administration routes can be parenteral administration, a mucosal administration, intravenous administration, depot injection, subcutaneous administration, topical administration, intradermal administration, oral administration, sublingual administration, intranasal administration, or intramuscular administration. The choice of administration route can depend on the compound identity (e.g., the physical and chemical properties of the compound) as well as the age and weight of the animal, the particular disease (e.g., fibrosis), and the severity of the disease (e.g., stage or severity of disease). Of course, combinations of administration routes can be administered, as desired.

Some embodiments of the invention include a method for providing a subject with a composition comprising one or more opioid receptor inhibitors (e.g., naltrexone) described herein (e.g., a pharmaceutical composition) which comprises one or more administrations of one or more such compositions; the compositions may be the same or different if there is more than one administration.

Diseases that can be treated in an animal (e.g., mammals, porcine, canine, avian (e.g., chicken), bovine, feline, primates, rodents, monkeys, rabbits, mice, rats, and humans) using one or more opioid receptor inhibitors include, but are not limited to fibrosis.

In some embodiments, fibrosis that can be treated in an animal (e.g., mammals, porcine, canine, avian (e.g., chicken), bovine, feline, primates, rodents, monkeys, rabbits, mice, rats, and humans) using an opioid receptor inhibitor include, but are not limited to lung fibrosis, pulmonary fibrosis, cystic fibrosis, idiopathic pulmonary fibrosis (IPF), or radiation-induced lung injury. In some embodiments, fibrosis that can be treated include, but are not limited to, lung fibrosis (e.g., pulmonary fibrosis, cystic fibrosis, idiopathic pulmonary fibrosis (IPF), radiation-induced lung injury, or radiation-induced lung injury resulting from treatment for cancer), skin fibrosis, kidney fibrosis, liver fibrosis (e.g., cirrhosis), heart fibrosis (e.g., atrial fibrosis, endomyocardial fibrosis, or myocardial infarction), brain fibrosis (e.g., glial scar), or other forms of fibrosis including but not limited to arterial stiffness, arthrofibrosis (e.g., knee, shoulder, or other joints), crohn's disease (e.g., intestine), dupuytren's contracture (e.g., hand or finger), keloid (e.g., skin), mediastinal fibrosis (e.g., soft tissue of the mediastinum), myelofibrosis (e.g., bone marrow), peyronie's disease (e.g., penis), nephrogenic systemic fibrosis (e.g., skin), progressive massive fibrosis (e.g., a complication of coal workers' pneumoconiosis), retroperitoneal fibrosis (e.g., soft tissue of the retroperitoneum), scleroderma/systemic sclerosis (e.g., skin or lung), adhesive capsulitis (e.g., shoulder), or other organ fibrosis. In other embodiments, fibrosis that can be treated can include lung fibrosis, kidney fibrosis, skin fibrosis, liver fibrosis, heart fibrosis, or brain fibrosis. In other embodiments, fibrosis that can be treated can include lung fibrosis, kidney fibrosis, liver fibrosis, heart fibrosis, or brain fibrosis. In other embodiments, fibrosis that can be treated can include lung fibrosis, liver fibrosis, heart fibrosis, or brain fibrosis. In certain embodiments, fibrosis that can be treated can include lung fibrosis, pulmonary fibrosis, cystic fibrosis, idiopathic pulmonary fibrosis (IPF), radiation-induced lung injury, liver fibrosis, cirrhosis, heart fibrosis, atrial fibrosis, endomyocardial fibrosis, or myocardial infarction. In certain embodiments, fibrosis that can be treated can include lung fibrosis, pulmonary fibrosis, cystic fibrosis, idiopathic pulmonary fibrosis (IPF), radiation-induced lung injury, skin fibrosis, kidney fibrosis, liver fibrosis, cirrhosis, heart fibrosis, atrial fibrosis, endomyocardial fibrosis, or myocardial infarction. In certain embodiments, fibrosis that can be treated can include lung fibrosis, pulmonary fibrosis, cystic fibrosis, idiopathic pulmonary fibrosis (IPF), radiation-induced lung injury, kidney fibrosis, liver fibrosis, cirrhosis, heart fibrosis, atrial fibrosis, endomyocardial fibrosis, or myocardial infarction. In other embodiments, fibrosis that can be treated can include lung fibrosis, pulmonary fibrosis, cystic fibrosis, idiopathic pulmonary fibrosis (IPF), radiation-induced lung injury, skin fibrosis, kidney fibrosis, heart fibrosis, atrial fibrosis, endomyocardial fibrosis, or myocardial infarction. In other embodiments, fibrosis that can be treated can include lung fibrosis, pulmonary fibrosis, cystic fibrosis, idiopathic pulmonary fibrosis (IPF), radiation-induced lung injury, kidney fibrosis, heart fibrosis, atrial fibrosis, endomyocardial fibrosis, or myocardial infarction. In other embodiments, fibrosis that can be treated can include lung fibrosis, pulmonary fibrosis, cystic fibrosis, idiopathic pulmonary fibrosis (IPF), radiation-induced lung injury, heart fibrosis, atrial fibrosis, endomyocardial fibrosis, or myocardial infarction. In other embodiments, fibrosis that can be treated can include lung fibrosis, pulmonary fibrosis, cystic fibrosis, idiopathic pulmonary fibrosis (IPF), or radiation-induced lung injury. In other embodiments, the fibrosis that is treated is not liver fibrosis. In other embodiments, the fibrosis that is treated is not cirrhosis.

Animals that can be treated include but are not limited to mammals, rodents, primates, monkeys (e.g., macaque, rhesus macaque, pig tail macaque), humans, canine, feline, porcine, avian (e.g., chicken), bovine, mice, rabbits, and rats. As used herein, the term “subject” refers to both human and animal subjects. In some instances, the animal is in need of the treatment (e.g., by showing signs of disease or fibrosis).

In some embodiments, fibrosis that can be treated in an animal (e.g., mammals, porcine, canine, avian (e.g., chicken), bovine, feline, primates, rodents, monkeys, rabbits, mice, rats, and humans) using one or more opioid receptor inhibitors include, but are not limited to fibrosis that can be treated by inhibiting (e.g., reducing the activity or expression of) a mu opioid receptor (MOR), a kappa opioid receptor (KOR), a delta opioid receptor (DOR), or combinations thereof. In some embodiments, fibrosis that can be treated in an animal include fibrosis that can be treated by inhibiting MOR, KOR or both.

As used herein, the term “treating” (and its variations, such as “treatment”) is to be considered in its broadest context. In particular, the term “treating” does not necessarily imply that an animal is treated until total recovery. Accordingly, “treating” includes amelioration of the symptoms, relief from the symptoms or effects associated with a condition, decrease in severity of a condition, or preventing, preventively ameliorating symptoms, or otherwise reducing the risk of developing a particular condition. As used herein, reference to “treating” an animal includes but is not limited to prophylactic treatment and therapeutic treatment. Any of the compositions (e.g., pharmaceutical compositions) described herein can be used to treat an animal.

As related to treating fibrosis (e.g., lung fibrosis, pulmonary fibrosis, cystic fibrosis, idiopathic pulmonary fibrosis (IPF), or radiation-induced lung injury), treating can include but is not limited to prophylactic treatment and therapeutic treatment. As such, treatment can include, but is not limited to: preventing fibrosis (e.g., lung fibrosis, pulmonary fibrosis, cystic fibrosis, idiopathic pulmonary fibrosis (IPF), or radiation-induced lung injury); reducing the risk of fibrosis (e.g., lung fibrosis, pulmonary fibrosis, cystic fibrosis, idiopathic pulmonary fibrosis (IPF), or radiation-induced lung injury); ameliorating or relieving symptoms of fibrosis (e.g., lung fibrosis, pulmonary fibrosis, cystic fibrosis, idiopathic pulmonary fibrosis (IPF), or radiation-induced lung injury); eliciting a bodily response against fibrosis (e.g., lung fibrosis, pulmonary fibrosis, cystic fibrosis, idiopathic pulmonary fibrosis (IPF), or radiation-induced lung injury); inhibiting the development or progression of fibrosis (e.g., lung fibrosis, pulmonary fibrosis, cystic fibrosis, idiopathic pulmonary fibrosis (IPF), or radiation-induced lung injury); inhibiting or preventing the onset of symptoms associated with fibrosis (e.g., lung fibrosis, pulmonary fibrosis, cystic fibrosis, idiopathic pulmonary fibrosis (IPF), or radiation-induced lung injury); reducing the severity of fibrosis (e.g., lung fibrosis, pulmonary fibrosis, cystic fibrosis, idiopathic pulmonary fibrosis (IPF), or radiation-induced lung injury); causing a regression of fibrosis (e.g., lung fibrosis, pulmonary fibrosis, cystic fibrosis, idiopathic pulmonary fibrosis (IPF), or radiation-induced lung injury) or one or more of the symptoms associated with fibrosis (e.g., a decrease in the amount of fibrosis); causing remission of fibrosis (e.g., lung fibrosis, pulmonary fibrosis, cystic fibrosis, idiopathic pulmonary fibrosis (IPF), or radiation-induced lung injury); or preventing relapse of fibrosis (e.g., lung fibrosis, pulmonary fibrosis, cystic fibrosis, idiopathic pulmonary fibrosis (IPF), or radiation-induced lung injury). In some embodiments, treating does not include prophylactic treatment of fibrosis (e.g., preventing or ameliorating future fibrosis). In some embodiments, treating does not include prophylactic treatment of liver fibrosis.

Treatment of an animal (e.g., human) can occur using any suitable administration method (such as those disclosed herein) and using any suitable amount of a compound of an opioid receptor inhibitor (e.g., naltrexone). In some embodiments, methods of treatment comprise treating an animal for fibrosis (e.g., lung fibrosis, pulmonary fibrosis, cystic fibrosis, idiopathic pulmonary fibrosis (IPF), or radiation-induced lung injury). Some embodiments of the invention include a method for treating a subject (e.g., an animal such as a human or primate) with a composition comprising one or more opioid receptor inhibitors (e.g., naltrexone) (e.g., a pharmaceutical composition) which comprises one or more administrations of one or more such compositions; the compositions may be the same or different if there is more than one administration.

In some embodiments, the method of treatment includes administering an effective amount of a composition comprising one or more opioid receptor inhibitors (e.g., naltrexone). As used herein, the term “effective amount” refers to a dosage or a series of dosages sufficient to affect treatment (e.g., to treat fibrosis, such as but not limited to lung fibrosis, pulmonary fibrosis, cystic fibrosis, idiopathic pulmonary fibrosis (IPF), or radiation-induced lung injury) in an animal. In some embodiments, an effective amount can encompass a therapeutically effective amount, as disclosed herein. In certain embodiments, an effective amount can vary depending on the subject and the particular treatment being affected. The exact amount that is required can, for example, vary from subject to subject, depending on the age and general condition of the subject, the particular adjuvant being used (if applicable), administration protocol, and the like. As such, the effective amount can, for example, vary based on the particular circumstances, and an appropriate effective amount can be determined in a particular case. An effective amount can, for example, include any dosage or composition amount disclosed herein. In some embodiments, an effective amount of one or more opioid receptor inhibitors (for example, but not limited to naltrexone) (which can be administered to an animal such as mammals, primates, monkeys or humans) can be an amount of about 0.005 to about 50 mg/kg body weight, about 0.005 to about 80 mg/kg body weight, about 0.005 to about 100 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some embodiments, the dosage can be about 0.5 mg/kg human body weight, about 5 mg/kg human body weight, about 6.5 mg/kg human body weight, about 10 mg/kg human body weight, about 50 mg/kg human body weight, about 80 mg/kg human body weight, or about 100 mg/kg human body weight. In some instances, an effective amount of one or more opioid receptor inhibitors (for example, but not limited to naltrexone) (which can be administered to an animal such as mammals, rodents, mice, rabbits, feline, porcine, or canine) can be an amount of about 0.005 to about 50 mg/kg body weight, about 0.005 to about 100 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg. In some embodiments, an effective amount of one or more opioid receptor inhibitors (for example, but not limited to naltrexone) (which can be administered to an animal such as mammals, primates, monkeys or humans) can be an amount of about 1 to about 1000 mg/kg body weight, about 5 to about 500 mg/kg body weight, about 10 to about 200 mg/kg body weight, about 25 to about 100 mg/kg body weight, about 1 mg/kg, about 2 mg/kg, about 5 mg/kg, about 10 mg/kg, about 25 mg/kg, about 50 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg. In regard to some conditions, the dosage can be about 5 mg/kg human body weight, about 10 mg/kg human body weight, about 20 mg/kg human body weight, about 80 mg/kg human body weight, or about 100 mg/kg human body weight. In some instances, an effective amount of one or more opioid receptor inhibitors (for example, but not limited to naltrexone) (which can be administered to an animal such as mammals, rodents, mice, rabbits, feline, porcine, or canine) can be an amount of about 1 to about 1000 mg/kg body weight, about 5 to about 500 mg/kg body weight, about 10 to about 200 mg/kg body weight, about 25 to about 100 mg/kg body weight, about 1 mg/kg, about 2 mg/kg, about 5 mg/kg, about 10 mg/kg, about 25 mg/kg, about 50 mg/kg, about 80 mg/kg, about 100 mg/kg, about 150 mg/kg, about 200 mg/kg, about 300 mg/kg, about 400 mg/kg, about 500 mg/kg, about 600 mg/kg, about 700 mg/kg, about 800 mg/kg, about 900 mg/kg, or about 1000 mg/kg.

“Therapeutically effective amount” means an amount effective to achieve a desired and/or beneficial effect (e.g., decreasing amount of fibrosis). A therapeutically effective amount can be administered in one or more administrations. For some purposes of this invention, a therapeutically effective amount is an amount appropriate to treat an indication (e.g., to treat fibrosis). By treating an indication is meant achieving any desirable effect, such as one or more of palliate, ameliorate, stabilize, reverse, slow, or delay disease (e.g., fibrosis) progression, increase the quality of life, or to prolong life. Such achievement can be measured by any suitable method, such as but not limited to measurement of the amount of fibrosis, the number of fibrocytes, the number of fibroblasts, the number of myofibroblasts, the extent of subpleural lung thickening, lung weight, body weight, lung function, or any suitable method to assess the progression of pulmonary fibrosis.

In some embodiments, other fibrosis treatments are optionally included, and can be used with the inventive treatments described herein (e.g., administering opioid receptor inhibitors). Other fibrosis treatments can include any known fibrosis treatment that is suitable to treat fibrosis. Examples of known fibrosis treatments include but are not limited to administration of: antibiotics (e.g., penicillins, methicillin, oxacillin, nafcillin, cabenicillin, ticarcillin, piperacillin, mezlocillin, azlocillin, ticarcillin clavulanic acid, piperacillin tazobactam, cephalosporins, cephalexin, cefdinir, cefprozil, cefaclor, cefepime, sulfa, sulfamethoxazole, trimethoprim, erythromycin/sulfisoxazole, macrolides, erythromycin, clarithromycin, azithromycin, tetracyclines, tetracycline, doxycycline, minocycline, tigecycline, vancomycin, imipenem, meripenem, colistimethate/colistin, aminoglycosides, tobramycin, amikacin, gentamicin, quinolones, aztreonam, or linezolid), anti-inflammatory drugs (e.g., NSAIDs, aspirin, ibuprofen, naproxen, corticosteroids, cortisol, corticosterone, cortisone, or aldosterone), bronchodilators (e.g., albuterol or levalbuterol hydrochloride), mucus thinners (e.g., hypertonic saline or Dornase alfa), antifibrotic medications (e.g., pirfenidone, nintedanib, N-acetylcysteine, ivacaftor, or lumacaftor/ivacaftor). Other fibrosis treatment can also include administering a non-drug respiratory therapy such as but not limited to airway clearance techniques (e.g., postural drainage and chest percussion, exercise, breathing exercises, or use of mechanical equipment such as high-frequency chest compression vest or positive expiratory pressure therapy). Other fibrosis treatment can also include organ transplantation (e.g., lung, skin, kidney, liver, or heart).

In some embodiments, administration of pirfenidone, nintedanib, or both can be used as part of the treatment regime (i.e., in addition to administration of one or more opioid receptor inhibitors and as an other fibrosis treatment); administration of pirfenidone, nintedanib, or both, can include separate administrations (i.e., in a separate composition from the opioid receptor inhibitor) or can be added to the composition comprising the opioid receptor inhibitor.

In some embodiments, additional optional treatments (e.g., as an other fibrosis treatment) can also include one or more of surgical intervention, hormone therapies, immunotherapy, and adjuvant systematic therapies.

### Opioid Receptor Inhibitors

In some embodiments of the invention, any suitable opioid receptor can be used in the methods described herein, including but not limited methods for treating fibrosis (e.g., lung fibrosis, pulmonary fibrosis, cystic fibrosis, idiopathic pulmonary fibrosis (IPF), radiation-induced lung injury, or radiation-induced lung injury resulting from treatment for cancer).

In some embodiments, opioid receptor inhibitors can inhibit (e.g., fully inhibit or partially inhibit) one or more opioid receptors by, for example, reducing the activity or expression of an opioid receptor. In other embodiments, opioid receptor inhibitors can be opioid receptor antagonists, opioid receptor partial antagonists, opioid receptor inverse agonists, opioid receptor partial inverse agonists, or combinations thereof. In certain embodiments, inhibition can occur using any suitable mechanism, such as but not limited to blockading the receptor (e.g., partially or fully blocking other molecules from accessing one or more receptor sites), an antagonist mechanism, a partial antagonist mechanism, an inverse agonist mechanism, a partial inverse agonist mechanism, or a combination thereof.

In some embodiments, opioid receptors that can be inhibited include any suitable opioid receptor that can be inhibited to treat fibrosis. In other embodiments, opioid receptors that can be inhibited include, but are not limited to delta opioid receptors (e.g., deltal or delta 2), kappa opioid receptors (e.g., kappa1, kappa2, or kappa3), mu opioid receptors (e.g., mu1, mu2, or mu3), nociceptin opioid receptors, zeta opioid receptors, sigma opioid receptors, epsilon opioid receptors, or a combination thereof.

In some embodiments, the opioid receptor inhibitor can include any suitable opioid receptor inhibitor to treat fibrosis (e.g., lung fibrosis, pulmonary fibrosis, cystic fibrosis, or idiopathic pulmonary fibrosis (IPF)). In other embodiments, the opioid receptor inhibitors can include a mu opioid receptor (MOR) antagonist, an MOR partial antagonist, an MOR inverse agonist, an MOR partial inverse agonist, a kappa opioid receptor (KOR) antagonist, a KOR partial antagonist, a KOR inverse agonist, a KOR partial inverse agonist, a delta opioid receptor (DOR) antagonist, a DOR partial antagonist, a DOR inverse agonist, a DOR partial inverse agonist, a nociceptin opioid receptor inhibitor, a zeta opioid receptor inhibitor, a sigma opioid receptor inhibitor, or a epsilon opioid receptor inhibitor. In other embodiments, the opioid receptor inhibitors can include an MOR (e.g., mu1, mu2, or mu3) antagonist, an MOR (e.g., mu1, mu2, or mu3) partial antagonist, an MOR (e.g., mu1, mu2, or mu3) inverse agonist, an MOR (e.g., mu1, mu2, or mu3) partial inverse agonist, a KOR (e.g., kappa1, kappa2, or kappa3) antagonist, a KOR (e.g., kappa1, kappa2, or kappa3) partial antagonist, a KOR (e.g., kappa1, kappa2, or kappa3) inverse agonist, a KOR (e.g., kappa1, kappa2, or kappa3) partial inverse agonist, a DOR (e.g., deltal or delta2) antagonist, a DOR (e.g., deltal or delta2) partial antagonist, a DOR (e.g., deltal or delta2) inverse agonist, a DOR (e.g., deltal or delta2) partial inverse agonist, or a combination thereof.

In some embodiments, the opioid receptor inhibitor can be one or more of alvimopan, AT-076 (i.e., (3R)-7-hydroxy-N-[2S)-1-[4-(3-hydroxyphenyl)piperidin-1-yl]-3-methylbutan-2-yl]-1,2,3,4-tetrahydroisoquinoline-3-carboxamide), axelopran, bevenopran, buprenorphine (e.g., as the combination of buprenorphine & samidorphan or the combination of buprenorphine & naltrexone), butorphanol, CERC-501 (i.e., 4-(4-{[2S)-2-(3,5-Dimethylphenyl)-1-pyrrolidinyl]methyl}phenoxy)-3-fluorobenzamide; CAS number 1174130-61-0), cyprodime, dezocine, diprenorphine, eptazocine, J-113,397 (i.e., 1-[(3R,4R)-1-cyclooctylmethyl-3-hydroxymethyl-4-piperidyl]-3-ethyl-1, 3-dihydro-2H-benzimidazol-2-one; CAS number 256640-45-6) or a racemic mixture thereof, JDTic (i.e., (3R)-7-Hydroxy-N-R2S)-1-[(3R,4R)-4-(3-hydroxyphenyl)-[3,4-dimethylpiperidin-1-yl]-3-methylbutan-2-yl]-1,2,3,4-tetrahydroisoquinoline-3-carboxamide; CAS number 361444-66-8), JTC-801 (i.e., N-(4-amino-2-methylquinolin-6-yl)-2-[(4-ethylphenoxy)methyl]benzamide; CAS number 244218-51-7), levallorphan, levorphanol, LY-2940094 (i.e., 2-[4-[(2-chloro-4,4-difluoro-spiro[5H-thieno [2,3-c]pyran-7,4′-piperidine]-1′-yl)methyl]-3-methyl-pyrazol-1-yl]-3-pyridyflmethanol; CAS number 1307245-86-8), methylnaltrexone, methylsamidorphan, nalbuphine, naldemedine, nalmefene, nalodeine, nalorphine, nalorphine dinicotinate, naloxegol, naloxone, 6β-naltrexol, naltrexone (i.e., 17-(cyclopropylmethyl)-4,5α-epoxy- 3,14-dihydroxymorphinan-6-one; CAS number 16590-41-3), naltrindole, norbinaltorphimine, pentazocine, PF-4455242 (i.e., 2-Methyl-N-{[2′-(1-pyrrolidinylsulfonyl)-4-biphenylyl]methyl}-1-propanamine; CAS number 1202647-54-8), phenazocine, SB-612,111 (i.e., (5S,7S)-7-{[442,6-dichlorophenyl)piperidin-1-yl]methyl}-1-methyl-6,7,8,9-tetrahydro-5H-benzo[7]annulen-5-ol; CAS number 371980-98-2), or samidorphan; or a salt, ester, or solvate of any of the aforementioned. In some embodiments, the opioid receptor inhibitor can be one or more of diprenorphine, levallorphan, nalmefene, nalorphine, nalorphine dinicotinate, naloxone, naltrexone, or samidorphan; or a salt, ester, or solvate of any of the aforementioned. In some embodiments, the opioid receptor inhibitor can be one or more of nalmefene, naloxone, naltrexone, or samidorphan; or a salt, ester, or solvate of any of the aforementioned. In some embodiments, the opioid receptor inhibitor can be naltrexone.

In some embodiments, the opioid receptor inhibitor can be in the form of a salt, an ester, or a solvate. In other embodiments, the opioid receptor inhibitor can be in various forms, such as uncharged molecules, components of molecular complexes, or non-irritating pharmacologically acceptable salts, including but not limited to hydrochloride, hydrobromide, sulphate, phosphate, nitrate, borate, acetate, maleate, tartrate, and salicylate. In some instances, for acidic compounds, salts can include metals, amines, or organic cations (e.g. quaternary ammonium). Esters can include any suitable esters such as but not limited to when an —OH group is replaced by an —O-alkyl group, where alkyl can be but is not limited to methyl, ethyl, propyl, or butyl. Solvates can include any suitable solvent (e.g., water, alcohols, ethanol) complexed (e.g., reversibly associated) with the molecule (e.g., opioid receptor inhibitor).

### Compositions Used for Treating

In certain embodiments, one or more opioid receptor inhibitors (e.g., naltrexone) can be part of a composition and can be in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, or no more than about 99.99%, from about 0.0001% to about 99%, from about 0.0001% to about 50%, from about 0.01% to about 95%, from about 1% to about 95%, from about 10% to about 90%, or from about 25% to about 75%.

In some embodiments, one or more opioid receptor inhibitors (e.g., naltrexone) can be purified or isolated in an amount (by weight of the total composition) of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.0001% to about 99%, from about 0.0001% to about 50%, from about 0.01% to about 95%, from about 1% to about 95%, from about 10% to about 90%, or from about 25% to about 75%.

Some embodiments of the present invention include compositions comprising one or more opioid receptor inhibitors (e.g., naltrexone). In certain embodiments, the composition is a pharmaceutical composition, such as compositions that are suitable for administration to animals (e.g., mammals, primates, monkeys, humans, canine, feline, porcine, mice, rabbits, or rats). In some instances, the pharmaceutical composition is non-toxic, does not cause side effects, or both. In some embodiments, there may be inherent side effects (e.g., it may harm the patient or may be toxic or harmful to some degree in some patients).

“Therapeutically effective amount” means an amount effective to achieve a desired and/or beneficial effect. An effective amount can be administered in one or more administrations. For some purposes of this invention, a therapeutically effective amount is an amount appropriate to treat an indication. By treating an indication is meant achieving any desirable effect, such as one or more of palliate, ameliorate, stabilize, reverse, slow, or delay disease progression, increase the quality of life, or to prolong life. Such achievement can be measured by any suitable method, such as measurement of the amount of fibrosis, the number of fibrocytes, the number of fibroblasts, the number of myofibroblasts, the extent of subpleural lung thickening, lung weight, body weight, lung function, or any suitable method to assess the progression of pulmonary fibrosis.

In some embodiments, one or more opioid receptor inhibitors (e.g., naltrexone) can be part of a pharmaceutical composition and can be in an amount of at least about 0.0001%, at least about 0.001%, at least about 0.10%, at least about 0.15%, at least about 0.20%, at least about 0.25%, at least about 0.50%, at least about 0.75%, at least about 1%, at least about 10%, at least about 25%, at least about 50%, at least about 75%, at least about 90%, at least about 95%, at least about 99%, at least about 99.99%, no more than about 75%, no more than about 90%, no more than about 95%, no more than about 99%, no more than about 99.99%, from about 0.001% to about 99%, from about 0.001% to about 50%, from about 0.1% to about 99%, from about 1% to about 95%, from about 10% to about 90%, or from about 25% to about 75%. In some embodiments, the pharmaceutical composition can be presented in a dosage form which is suitable for the topical, subcutaneous, intrathecal, intraperitoneal, oral, parenteral, rectal, cutaneous, nasal, vaginal, or ocular administration route. In other embodiments, the pharmaceutical composition can be presented in a dosage form which is suitable for parenteral administration, a mucosal administration, intravenous administration, depot injection (e.g., solid or oil based), subcutaneous administration, topical administration, intradermal administration, oral administration, sublingual administration, intranasal administration, or intramuscular administration. The pharmaceutical composition can be in the form of, for example, tablets, capsules, pills, powders granulates, suspensions, emulsions, solutions, gels (including hydrogels), pastes, ointments, creams, plasters, drenches, delivery devices, suppositories, enemas, injectables, implants, sprays, aerosols or other suitable forms.

In some embodiments, the pharmaceutical composition can include one or more formulary ingredients. A “formulary ingredient” can be any suitable ingredient (e.g., suitable for the drug(s), for the dosage of the drug(s), for the timing of release of the drugs(s), for the disease, for the disease state, or for the delivery route) including, but not limited to, water (e.g., boiled water, distilled water, filtered water, pyrogen-free water, or water with chloroform), sugar (e.g., sucrose, glucose, mannitol, sorbitol, xylitol, or syrups made therefrom), ethanol, glycerol, glycols (e.g., propylene glycol), acetone, ethers, DMSO, surfactants (e.g., anionic surfactants, cationic surfactants, zwitterionic surfactants, or nonionic surfactants (e.g., polysorbates)), oils (e.g., animal oils, plant oils (e.g., coconut oil or arachis oil), or mineral oils), oil derivatives (e.g., ethyl oleate, glyceryl monostearate, or hydrogenated glycerides), excipients, preservatives (e.g., cysteine, methionine, antioxidants (e.g., vitamins (e.g., A, E, or C), selenium, retinyl palmitate, sodium citrate, citric acid, chloroform, or parabens, (e.g., methyl paraben or propyl paraben)), or combinations thereof. For example, a depot injection (e.g., solid or oil based) could include one or more formulary ingredients.

In certain embodiments, pharmaceutical compositions can be formulated to release the one or more opioid receptor inhibitors (e.g., naltrexone) substantially immediately upon the administration or any substantially predetermined time or time after administration. Such formulations can include, for example, controlled release formulations such as various controlled release compositions and coatings. For example, a depot injection (e.g., solid or oil based) could be used for a controlled release (e.g., of naltrexone), and in some instances, could be injected once per month (or once per day, once per week, once per three months, once per six months, or once per year).

Other formulations (e.g., formulations of a pharmaceutical composition) can, in certain embodiments, include those incorporating the drug (or control release formulation) into food, food stuffs, feed, or drink. For example, naltrexone could be administered orally once per day, twice per day, three times per day, once per two days, or once per week.

Some embodiments of the invention can include methods of treating an organism for fibrosis. In certain embodiments, treating comprises administering at least one opioid receptor inhibitor. In other embodiments, treating comprises administering at least one opioid receptor inhibitor to an animal that is effective to treat fibrosis. In some embodiments, a composition or pharmaceutical composition comprises at least one opioid receptor inhibitor which can be administered to an animal (e.g., mammals, primates, monkeys, or humans) in an amount of about 0.005 to about 100 mg/kg body weight, about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 0.5 mg/kg, about 1 mg/kg, about 3 mg/kg, about 5 mg/kg, about 5.5 mg/kg, about 6 mg/kg, about 6.5 mg/kg, about 7 mg/kg, about 7.5 mg/kg, about 8 mg/kg, about 10 mg/kg, about 12 mg/kg, or about 15 mg/kg. In regard to some conditions, the dosage can be about 0.5 mg/kg human body weight, about 5 mg/kg human body weight, about 6.5 mg/kg human body weight, about 10 mg/kg human body weight, about 50 mg/kg human body weight, about 80 mg/kg human body weight, or about 100 mg/kg human body weight. In some instances, some animals (e.g., mammals, mice, rabbits, feline, porcine, or canine) can be administered a dosage of about 0.005 to about 100 mg/kg body weight, about 0.005 to about 50 mg/kg body weight, about 0.01 to about 15 mg/kg body weight, about 0.1 to about 10 mg/kg body weight, about 0.5 to about 7 mg/kg body weight, about 0.005 mg/kg, about 0.01 mg/kg, about 0.05 mg/kg, about 0.1 mg/kg, about 1 mg/kg, about 5 mg/kg, about 10 mg/kg, about 20 mg/kg, about 30 mg/kg, about 40 mg/kg, about 50 mg/kg, about 80 mg/kg, about 100 mg/kg, or about 150 mg/kg. Of course, those skilled in the art will appreciate that it is possible to employ many concentrations in the methods of the present invention, and using, in part, the guidance provided herein, will be able to adjust and test any number of concentrations in order to find one that achieves the desired result in a given circumstance. In other embodiments, the opioid receptor inhibitor can be administered in combination with one or more other therapeutic agents to treat a given fibrosis.

In some embodiments, the compositions can include a unit dose of one or more opioid receptor inhibitors in combination with a pharmaceutically acceptable carrier and, in addition, can include other medicinal agents, pharmaceutical agents, carriers, adjuvants, diluents, and excipients. In certain embodiments, the carrier, vehicle or excipient can facilitate administration, delivery and/or improve preservation of the composition. In other embodiments, the one or more carriers, include but are not limited to, saline solutions such as normal saline, Ringer's solution, PBS (phosphate-buffered saline), and generally mixtures of various salts including potassium and phosphate salts with or without sugar additives such as glucose. Carriers can include aqueous and non-aqueous sterile injection solutions that can contain antioxidants, buffers, bacteriostats, bactericidal antibiotics, and solutes that render the formulation isotonic with the bodily fluids of the intended recipient; and aqueous and non-aqueous sterile suspensions, which can include suspending agents and thickening agents. In other embodiments, the one or more excipients can include, but are not limited to water, saline, dextrose, glycerol, ethanol, or the like, and combinations thereof. Nontoxic auxiliary substances, such as wetting agents, buffers, or emulsifiers may also be added to the composition. Oral formulations can include such normally employed excipients as, for example, pharmaceutical grades of mannitol, lactose, starch, magnesium stearate, sodium saccharine, cellulose, and magnesium carbonate.

The presently-disclosed subject matter is further illustrated by the following specific but non-limiting examples. The following examples may include compilations of data that are representative of data gathered at various times during the course of development and experimentation related to the present invention.

### EXAMPLES

Certain methods used below can be found in SONTAKE et al., “Hsp90 regulation of fibroblast activation in pulmonary fibrosis” (2017) JCI Insight, Vol. 2, Issue 4, Article e91454. <<https://doi.org/10.1172/jci.insight.91454>>(which is herein incorporated by reference in its entirety).

Identification of naltrexone as a candidate therapeutic for Idiopathic Pulmonary Fibrosis (IPF) using computational methods. We used a previously published transcriptomic data set (GSE53845) recorded from lung biopsies of 40 IPF patients and 8 healthy controls. IPF disease signatures were created based on mRNA expression data and a linear model was fit for each gene to estimate the effect of IPF disease status and gene-expression changes (log₂fold-changes) in diseased vs normal control samples. This IPF gene signature was queried against the LINCS database, an extensive expression profiling resource. This database is a catalog of gene-expression profiles collected from human cells treated with chemical and genetic perturbagens. Pattern-matching software (LAMB et al., “The connectivity map: Using gene-expression signatures to connect small molecules, genes, and disease” (2006) Science, Vol. 313, pp. 1929-1935) was used to identify small molecules whose gene-signatures were negatively connected to the IPF gene-signatures. An inhibitor of opioid receptor, naltrexone, was among the top FDA-approved drugs with possible anti-fibrotic and therapeutic potential for IPF that we identified. Currently, naltrexone is approved for treating alcohol and drug addiction.

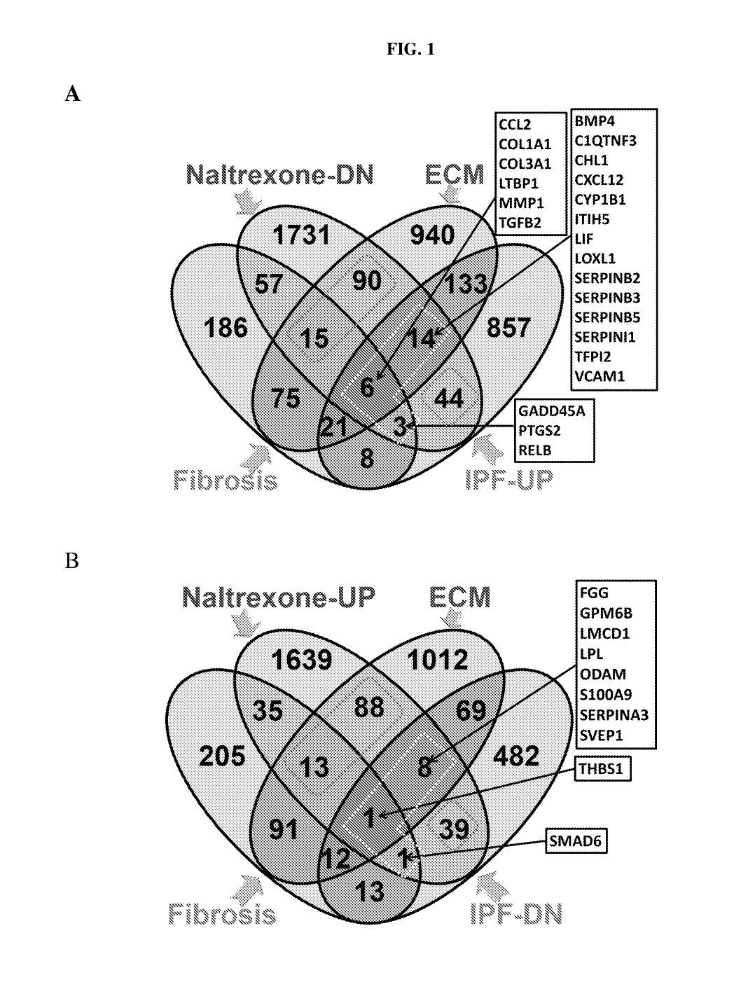

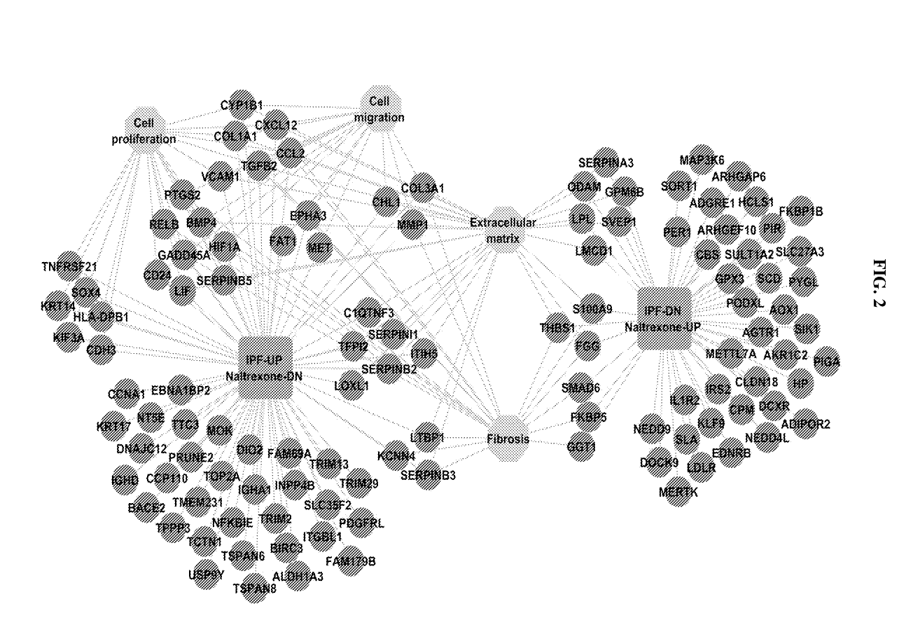

Connecting naltrexone targets and “off-targets” to IPF. We used two independently published gene-expression data sets from IPF patients (GSE53845 from DEPIANTO et al., “Heterogeneous gene expression signatures correspond to distinct lung pathologies and biomarkers of disease severity in idiopathic pulmonary fibrosis” (2015) Thorax., Vol. 70, pp. 48-56; and GSE32539 from YANG et al., “Expression of cilium-associated genes defines novel molecular subtypes of idiopathic pulmonary fibrosis” (2013) Thorax., Vol. 68, pp. 1114-1121) and compared them to naltrexone gene expression profiles (GEPs) and known Extracellular Matrix (ECM) and fibrogenic signaling pathway genes (FIG. 1). Genes upregulated in IPF lungs, but downregulated upon treatment with naltrexone (from NIH Library of Integrated Network-based Cellular Signatures—“LINCS”) showed several genes related to ECM production and fibrosis signaling, including CXCL12. As CXCL12 could interact with an opioid receptor (OPRM1), and CXCL12 appears to mediate traffic of fibrocytes to the lungs in the pathogenesis of pulmonary fibrosis, we conjectured that an antifibrotic mechanism of naltrexone could occur through modulation of OPRM1-CXCL12 interaction and that naltrexone could be used to pharmacologically manipulate CXCL12-mediated traffic of fibrocytes in IPF. To further investigate the role of naltrexone in IPF, we also performed an enrichment analysis of the negatively correlated gene sets between IPF lungs and naltrexone-treated cells from the LINCS database. The ToppFun application of the ToppGene Suite (CHEN et al., “Toppgene suite for gene list enrichment analysis and candidate gene prioritization” (2009) Nucleic Acids Res., Vol. 37, pp. W305-311.) was utilized to identify the most highly enriched biological processes common to both the IPF and naltrexone data sets. Surprisingly, ECM production, fibrosis, cell migration, and cell proliferation were the major biological processes that were down regulated by naltrexone in IPF [P<0.05; false discovery rate (FDR) corrected] (FIG. 2). More broadly, we also conjectured that opioid receptor antagonists (e.g., naltrexone) could be used to treat fibrosis (e.g., heart fibrosis, brain fibrosis, skin fibrosis, kidney fibrosis, liver fibrosis, lung fibrosis, or IPF).

Mouse model of TGFα-induced fibrosis. EGFR (HER1) belongs to a receptor tyrosine-kinase protein family that also includes HER2/neu, HERS, and HER4. Six EGFR ligands including Transforming Growth Factor alpha (TGFα) have been previously identified in lungs or lung cells. EGFR and its ligands are found in a number of cells in the lung, including the alveolar and airway epithelia, fibroblasts, and macrophages. In the lung, EGFR is reported to be activated both directly and indirectly by several inflammatory agents, including cytomegalovirus, endotoxin, tumor necrosis factor or TNF, and IL-13. Activation of EGFR is reported to regulate diverse cellular functions, some of which are associated with fibrogenesis and include cell growth, proliferation, differentiation, migration, and survival. In one study, TGFα was reported to be detected in the lung lavage fluid of all of 10 patients with IPF, but in none of 13 normal volunteers. Others reported an increase in TGFα and EGFR in IPF by immunohistochemistry with increased TGFα localized to Type II epithelial cells, fibroblasts, and the vascular endothelium compared with controls.

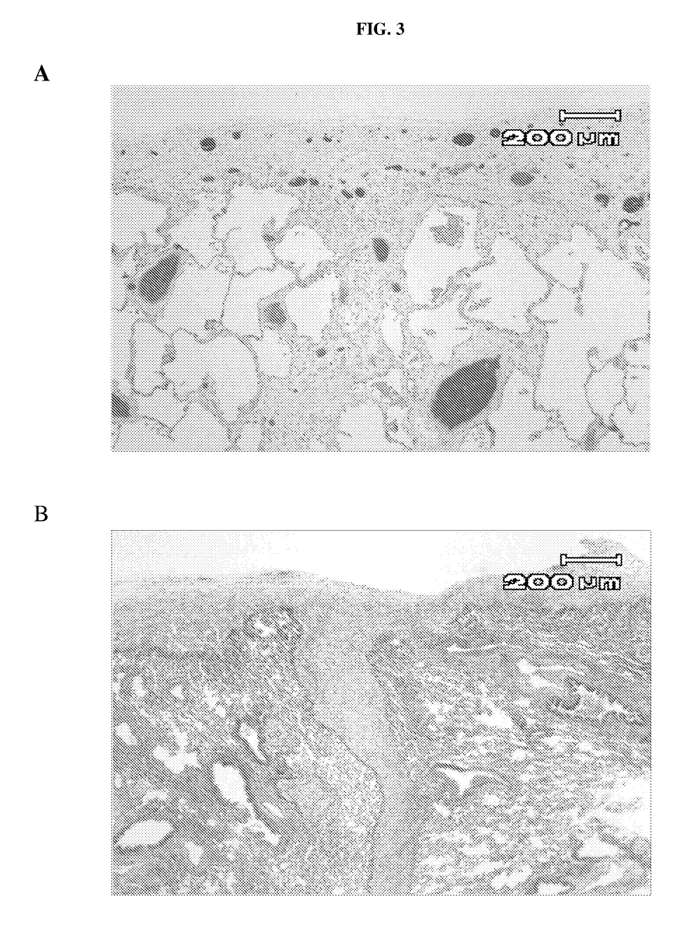

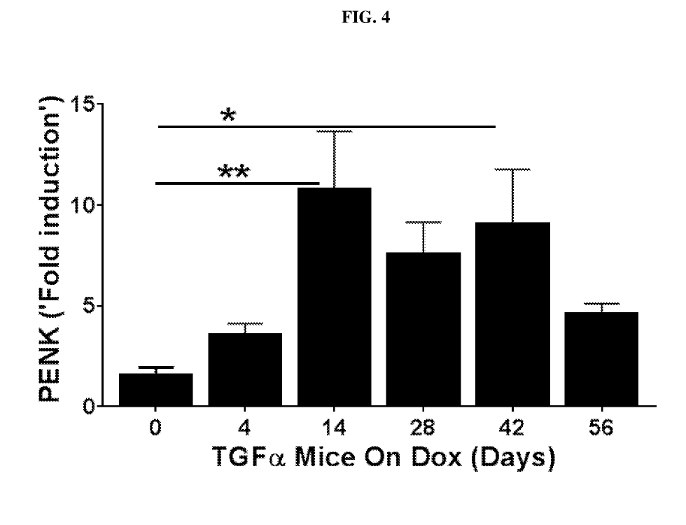

To probe mechanisms of EGFR-mediated lung remodeling, we generated transgenic mice in which TGFα was conditionally overexpressed in the lung epithelium using the CCSP rtTA promoter. When doxycycline (Dox) was administered, overexpression of TGFα in the adult mice (N=4−5 mice/group) caused progressive and extensive adventitial, interstitial, and subpleural fibrosis. Several histological features of fibrosis in the TGFα model were found in the pathologic lesions of IPF, including subpleural fibrosis radiating into the adjoining interstitium and the differentiation of myofibroblasts (FIG. 3). Physiologically, the mice developed progressive cachexia and lung-function decline with gene expression profiles similar to human IPF. The proenkephalin A (PENK) gene that codes for several opioid peptides was elevated in fibrotic lungs during TGFα-induced pulmonary fibrosis (FIG. 4). With these data, we have demonstrated that the TGFα transgenic mouse can be a useful model to, for example, assess the role of opioid-receptor signaling and the effect of opioid-receptor inhibitors in progressive pulmonary fibrosis.

Mouse model of bleomycin-induced fibrosis. Bleomycin is a nonribosomal antibiotic peptide isolated from Streptomyces verticillatus that has been used to treat multiple cancers. Bleomycin treatment induces DNA damage and reactive oxygen species generation. When lungs are exposed to bleomycin via the intratracheal route, mice develop lung injury and loss of the epithelial barrier that is marked by tissue inflammation and fibrosis. Bleomycin-driven fibrotic responses are short and reversible with limited or no significant changes in subpleural thickening and lung function.

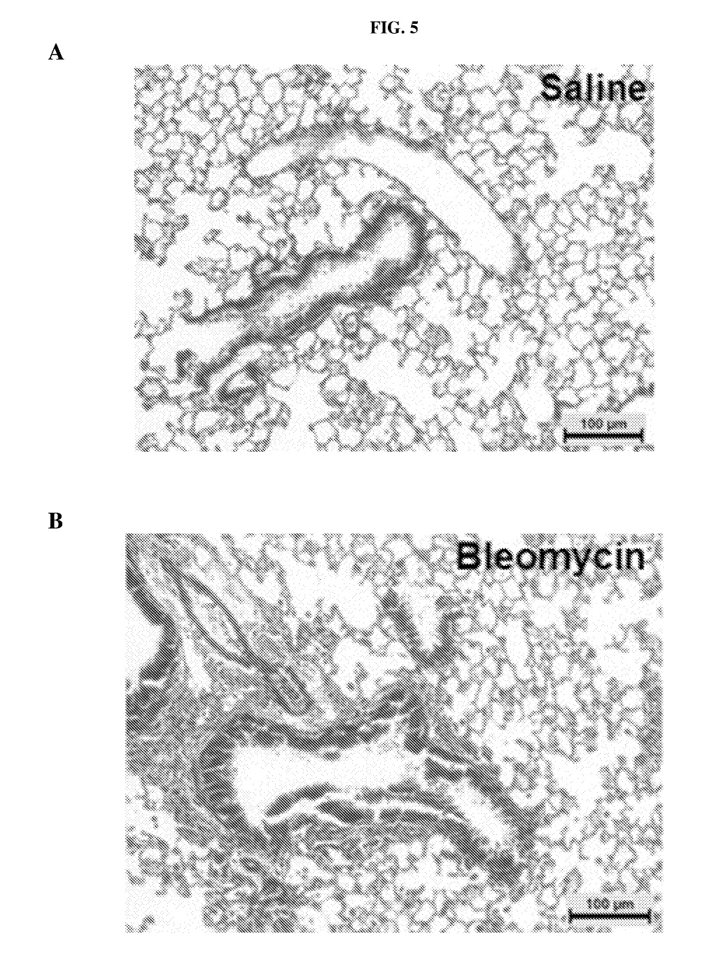

We developed an alternative mouse model of bleomycin-induced pulmonary fibrosis. For these experiments, we injected mice (N=5 mice/group) intradermally with bleomycin (6 units per kg body weight) for 5 days in a week for a total of 4 wks. The animals displayed a progressive decline in lung function with a greater than two-fold increase in airway resistance and lung hydroxyproline levels compared to saline-treated controls. Repetitive intradermal administration of bleomycin resulted in mild inflammation, but extensive fibrosis that persisted for several weeks in subpleural and parenchymal lung regions (FIG. 5). With these data, we have established a pre-clinical mouse model to, for example, test the effect of anti-fibrotic therapy with opioid receptor inhibitors (e.g., naltrexone) on established pulmonary fibrosis.

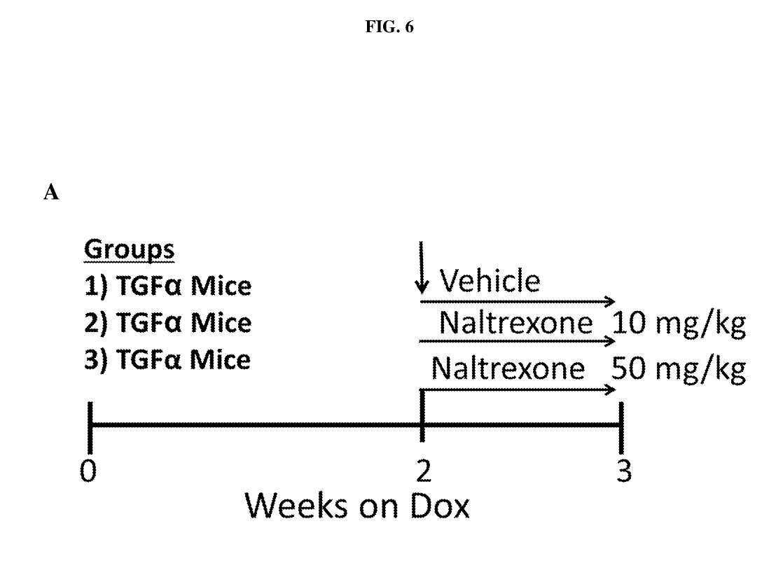

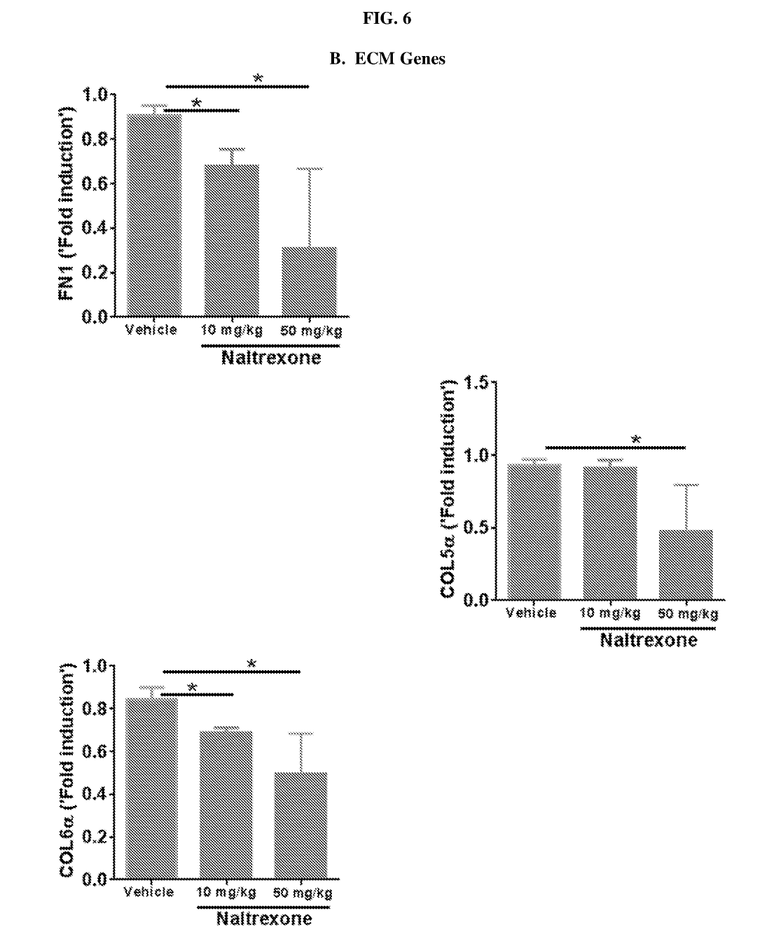

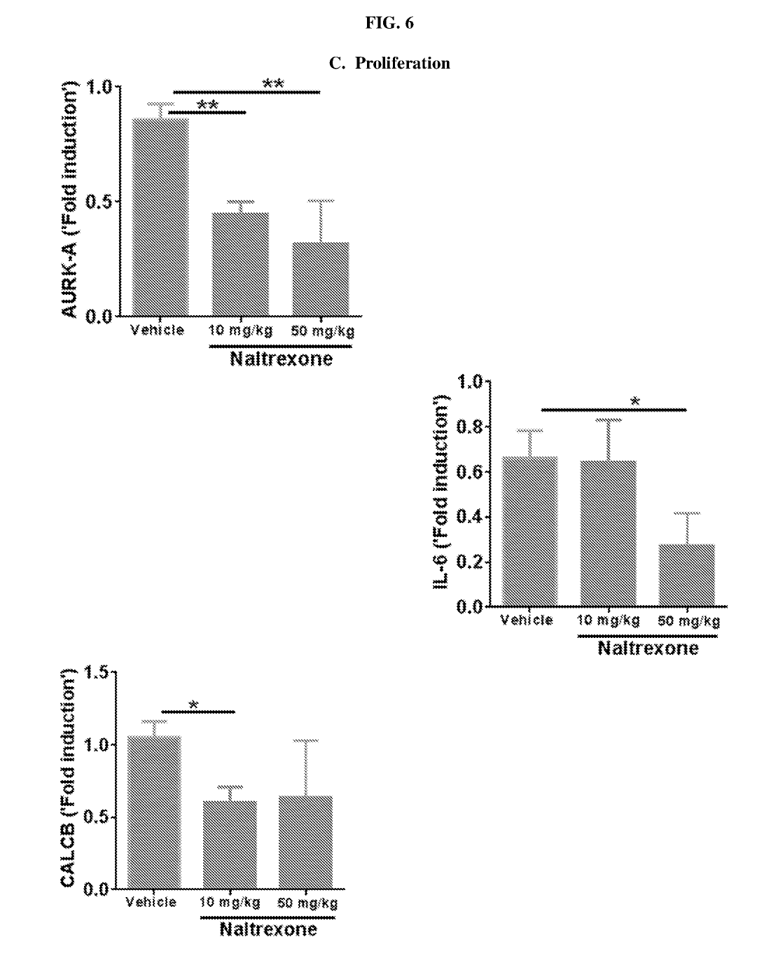

Naltrexone therapy attenuates ECM and proliferative gene expression in pulmonary fibrosis. FIG. 6A shows the experimental schemata of short-term naltrexone therapy study in vivo. Three groups of mice (N=4 mice/group) on Dox for two weeks were treated with either vehicle or naltrexone (10 mg/kg or 50 mg/kg bodyweight, b.i.d) and sacrificed after a week of treatment while continued on Dox. The down arrow represents initiation of the vehicle or drug treatment (at two weeks on Dox) and the horizontal arrows indicate the duration of vehicle or drug treatment (one week while continuing to administer Dox). FIG. 6B shows total lung RNA was analyzed for the expression of ECM genes: Fibronectin (FN1), Collagen alpha 1 V (Col5α), and Collagen alpha 1 VI (Col6α). The RNA from the three ECM genes decreased when treated with naltrexone. FIG. 6C shows total lung RNA was analyzed for the expression of proliferation genes: Aurora kinase A (Aurka), Interleuken 6 (IL-6), and Calcitonin Related Polypeptide Beta (Calcb). The RNA from the three proliferation genes decreased when treated with naltrexone.

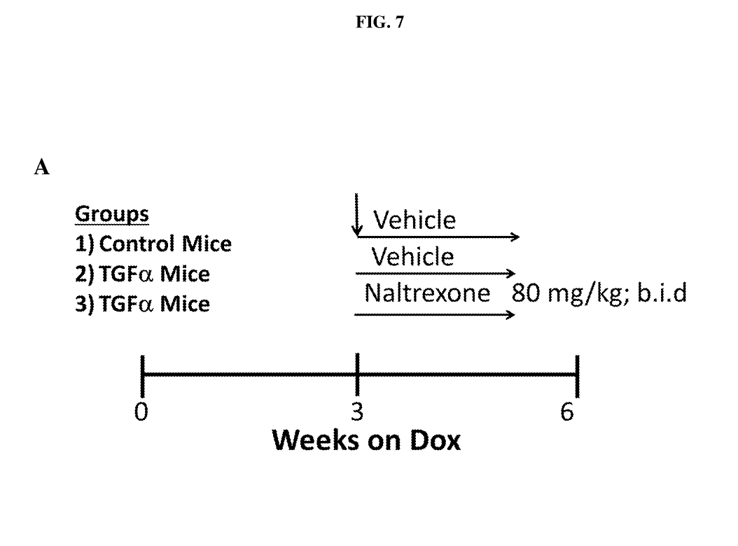

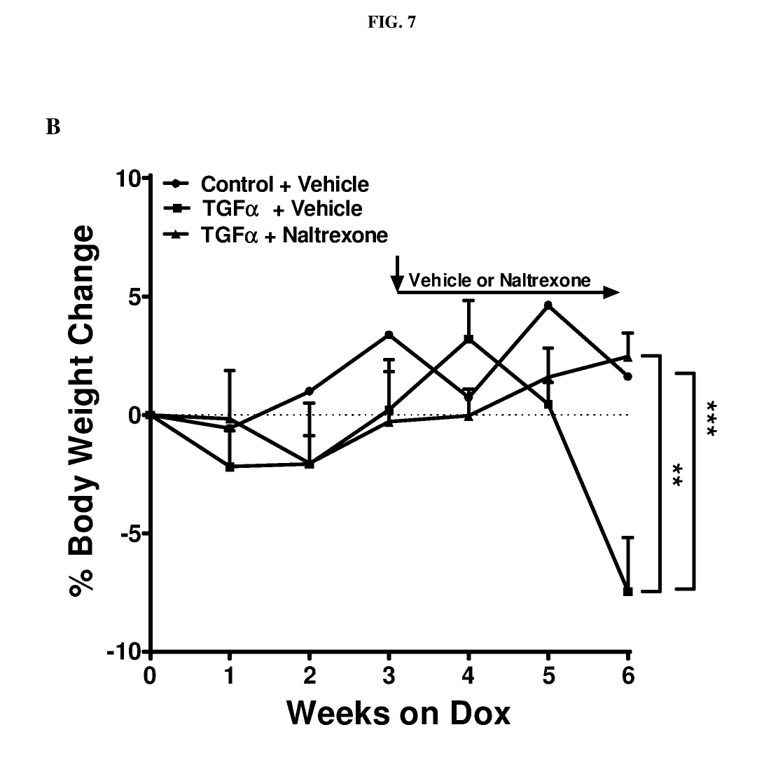

Naltrexone therapy attenuates body weight loss in pulmonary fibrosis. FIG. 7A shows experimental schemata of a naltrexone therapy study in vivo. Three groups of mice (N=4−6 mice/group) were on Dox for three weeks, and were then treated with either vehicle or naltrexone (80 mg/kg bodyweight, b.i.d) along with Dox for another three weeks; six weeks after being on DOX and three weeks after being treated with vehicle or naltrexone, the mice were sacrificed. The down arrow represents initiation of the vehicle or naltrexone treatment and the horizontal arrows indicate the duration of the vehicle or naltrexone treatment (three weeks while continuing to administer Dox). FIG. 7B shows the loss of body weight of the mice were attenuated by the naltrexone therapy compared vehicle treatment in TGFα mice (i.e., induced by Dox).

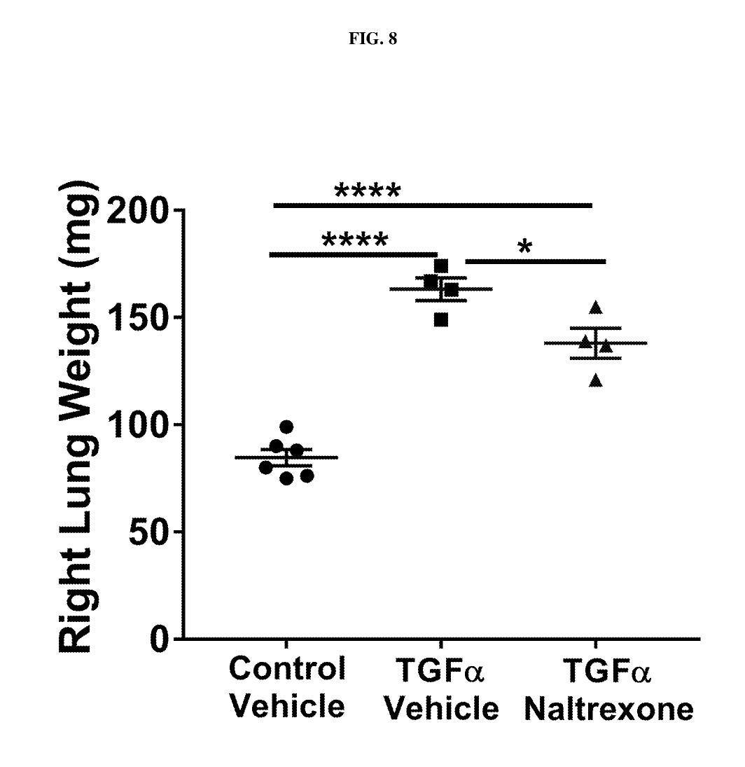

Naltrexone therapy attenuates increase in lung weights in pulmonary fibrosis. Mice (N=4−6 mice/group) were on Dox for three weeks, and were then treated with either vehicle or naltrexone (80 mg/kg bodyweight, b.i.d) along with Dox for another three weeks; six weeks after being on DOX and three weeks after being treated with vehicle or naltrexone, the mice were sacrificed (See, e.g., FIG. 7A). The results in FIG. 8 show that the increase in the lung weights were attenuated with naltrexone therapy compared vehicle treatment in TGFα mice (i.e., induced by Dox). In TGFα mice, increases in lung weight are due to pulmonary fibrosis.

Naltrexone therapy attenuates lung function decline in pulmonary fibrosis. Mice (N=4−6 mice/group) were on Dox for three weeks, and were then treated with either vehicle or naltrexone (80 mg/kg bodyweight, b.i.d) along with Dox for another three weeks; six weeks after being on DOX and three weeks after being treated with vehicle or naltrexone, the mice were sacrificed (See, e.g., FIG. 7A). The lung function measurements were performed using computer controlled FlexiVent system (SCIREQ Scientific Respiratory Equipment, Montreal, Quebec, Canada). Single frequency forced oscillation maneuver was performed to calculate the dynamics of tissue resistance, elastance, and compliance of the respiratory system (TANAKA et al., “Effects of Lecithinized Superoxide Dismutase and/or Pirfenidone Against Bleomycin-Induced Pulmonary Fibrosis” (2012) Chest, Vol. 142, No. 4, pp. 1011-1019; MADALA et al., “Dual Targeting of MEK and PI3K Pathways Attenuates Established and Progressive Pulmonary Fibrosis” (Jan. 2014) Plos One, Vol 9, Issue 1, article e86536, 11 pages—doi:10.1371/journal.pone.0086536). Measurement maneuvers executed by the flexiVent are referred to as perturbations. During a perturbation, an iso-volume ventilator compartment is established when the valves within the module close to the outside environment. The ventilator compartment consists of the subject's respiratory system, the cylinder, pathways outside the module by Y-tubing and pathways within the module, from the cylinder to the Y-tubing. Once the valves are closed, the perturbation, or forced oscillation, is applied to the ventilator compartment through movement of the piston. The signals generated during the perturbation are used to calculate parameters of respiratory mechanics that help to quantify fibrotic changes in the lungs (TANAKA et al., “Effects of Lecithinized Superoxide Dismutase and/or Pirfenidone Against Bleomycin-Induced Pulmonary Fibrosis” (2012) Chest, Vol. 142, No. 4, pp. 1011-1019; MADALA et al., “Dual Targeting of MEK and PI3K Pathways Attenuates Established and Progressive Pulmonary Fibrosis” (Jan. 2014) Plos One, Vol 9, Issue 1, article e86536, 11 pages—doi:10.1371/journal.pone.0086536; DE VLEESCHAUWER et al., “Repeated invasive lung function measurements in intubated mice: an approach for longitudinal lung research” (2011) Laboratory Animals, Vol. 45, Issue 2, pp. 81-89—DOI: 10.1258/1a.2010.010111). The lung function parameters measured using FlexiVent include: (1) Resistance (R)), a quantitative assessment of airway constriction in the lungs; (2) Elastance (E), a measure of the elastic rigidity or the stiffness in the lungs;(3) Compliance (C), the ease with which the lungs expand.

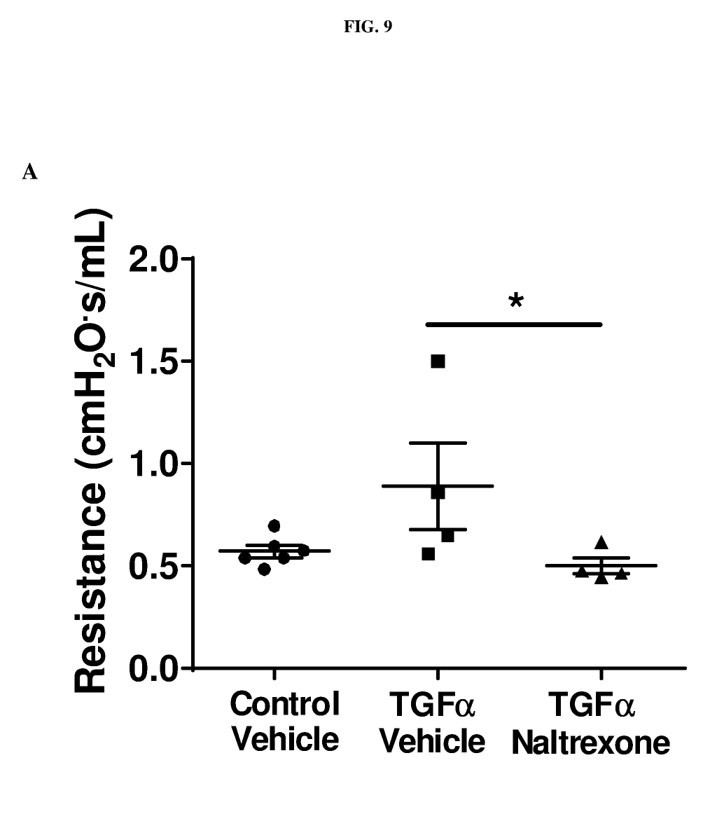

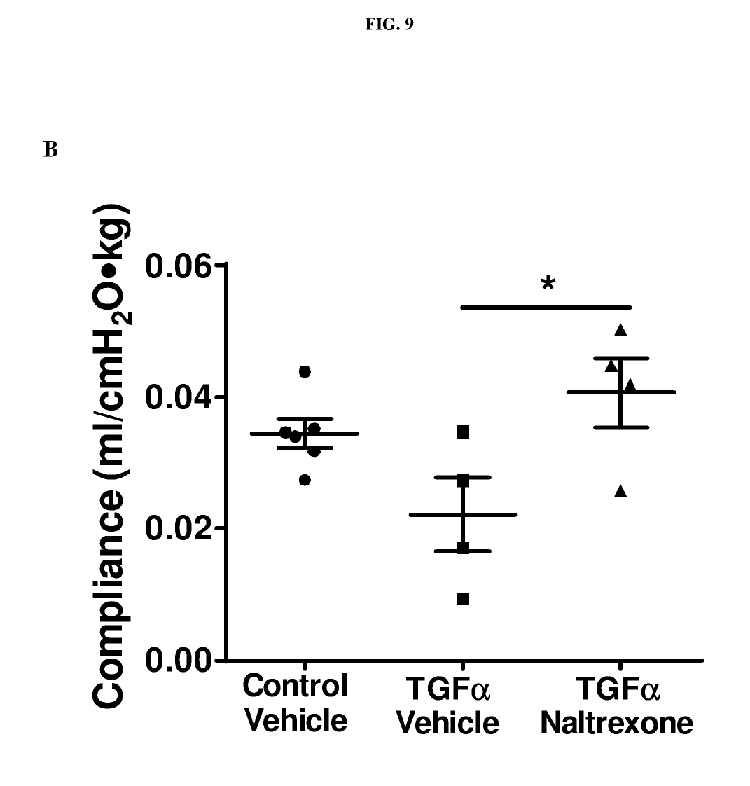

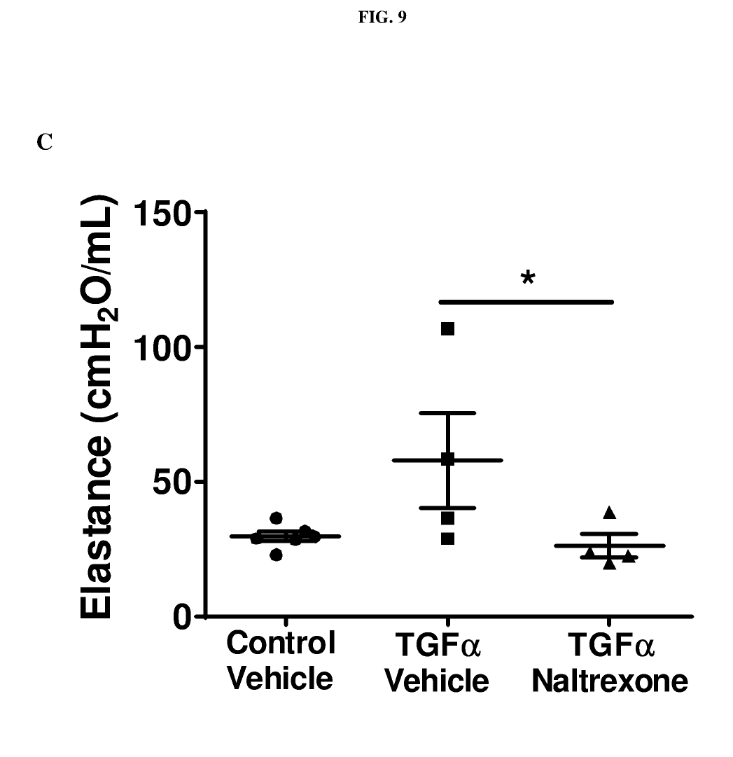

FIG. 9 shows that the decrease in the lung function was attenuated with naltrexone therapy compared vehicle treatment in TGFα mice. FIG. 9A shows that the Dox-induced changes in lung function (resistance and compliance) were reversed by the treatment with naltrexone. FIG. 9B shows that the Dox-induced changes in compliance were reversed by the treatment with naltrexone; compliance is the volume change that could be achieved in the lungs per unit pressure change. FIG. 9C shows that the Dox-induced changes in elastance were reversed by the treatment with naltrexone; elastance is the pressure change that is required to elicit a unit volume change.

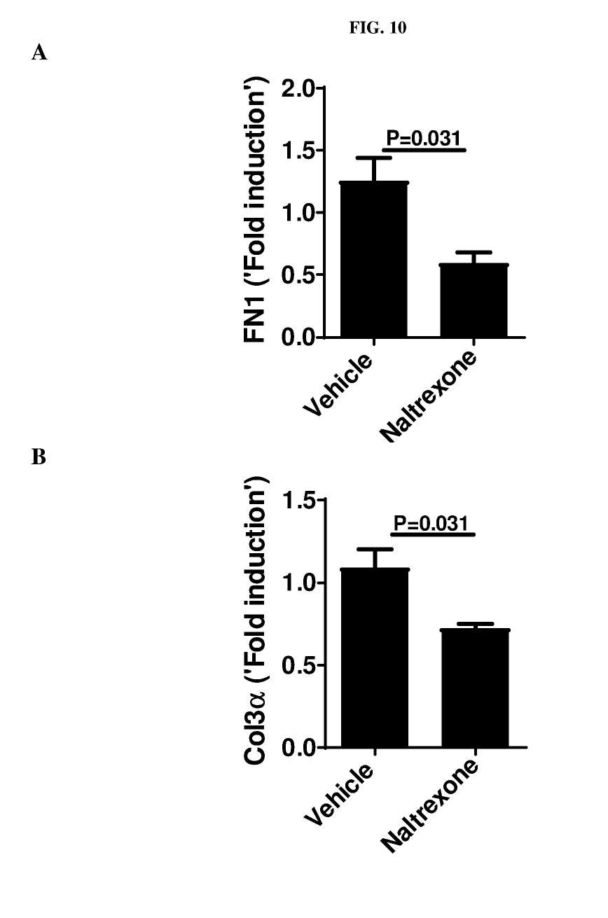

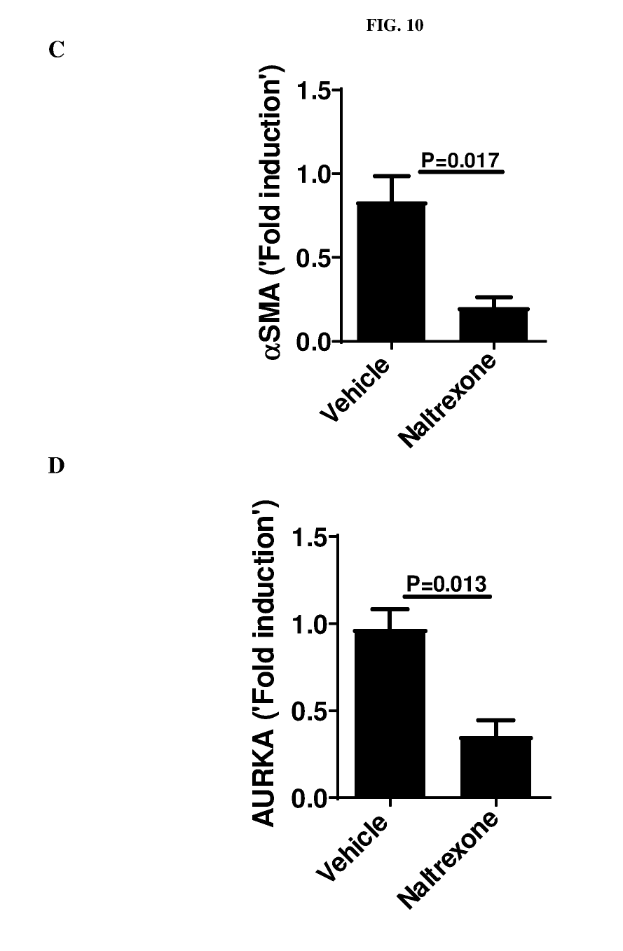

Naltrexone treatment inhibits IPF-specific genes involved in fibroproliferation and ECM production. We treated primary fibroblasts isolated from fibrotic lesions of IPF lungs. FIG. 10 shows that naltrexone inhibited the expression of genes involved in ECM deposition and fibroproliferation. In particular, FIG. 10A-C shows that the expression of ECM genes fibronectin 1 (FN1), procollagen 3α1 (Col3α), and alpha smooth-muscle actin (αSMA), respectively, were inhibited. FIG. 10D shows that the expression of proliferation gene Aurora kinase A (AURKA) was inhibited. These findings are consistent with a mechanism of action for opioid receptor inhibitors (e.g., naltrexone) that influences (e.g., decreases) ECM and proliferation.

The headings used in the disclosure are not meant to suggest that all disclosure relating to the heading is found within the section that starts with that heading. Disclosure for any subject may be found throughout the specification.

It is noted that terms like “preferably,” “commonly,” and “typically” are not used herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

As used in the disclosure, “a” or “an” means one or more than one, unless otherwise specified. As used in the claims, when used in conjunction with the word “comprising” the words “a” or “an” means one or more than one, unless otherwise specified. As used in the disclosure or claims, “another” means at least a second or more, unless otherwise specified. As used in the disclosure, the phrases “such as”, “for example”, and “e.g.” mean “for example, but not limited to” in that the list following the term (“such as”, “for example”, or “e.g.”) provides some examples but the list is not necessarily a fully inclusive list. The word “comprising” means that the items following the word “comprising” may include additional unrecited elements or steps; that is, “comprising” does not exclude additional unrecited steps or elements.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

Detailed descriptions of one or more embodiments are provided herein. It is to be understood, however, that the present invention may be embodied in various forms. Therefore, specific details disclosed herein (even if designated as preferred or advantageous) are not to be interpreted as limiting, but rather are to be used as an illustrative basis for the claims and as a representative basis for teaching one skilled in the art to employ the present invention in any appropriate manner Indeed, various modifications of the invention in addition to those described herein will become apparent to those skilled in the art from the foregoing description and the accompanying figures. Such modifications are intended to fall within the scope of the appended claims.

What is claimed is:

1. A method for treating an animal for fibrosis, comprising one or more administrations of one or more compositions comprising one or more opioid receptor inhibitors, wherein the compositions may be the same or different if there is more than one administration.

2. The method of claim 1, wherein one or more opioid receptor inhibitors inhibits one or more of a delta opioid receptor, deltal opioid receptor, delta2 opioid receptor, a kappa opioid receptor, kappa1 opioid receptor, kappa2 opioid receptor, kappa3 opioid receptor, a mu opioid receptor, mu1 opioid receptor, mu2 opioid receptor, mu3 opioid receptor, a nociceptin opioid receptor, a zeta opioid receptor, a sigma opioid receptor, or an epsilon opioid receptor.

3. The method of claim 1 or claim 2, wherein one or more opioid receptor inhibitors is a mu opioid receptor (MOR) antagonist, an MOR partial antagonist, an MOR inverse agonist, an MOR partial inverse agonist, a kappa opioid receptor (KOR) antagonist, a KOR partial antagonist, a KOR inverse agonist, a KOR partial inverse agonist, a delta opioid receptor (DOR) antagonist, a DOR partial antagonist, a DOR inverse agonist, a DOR partial inverse agonist, a nociceptin opioid receptor inhibitor, a zeta opioid receptor inhibitor, a sigma opioid receptor inhibitor, a epsilon opioid receptor inhibitor, or a combination thereof.

4. The method of any of claims 1-3, wherein one or more opioid receptor inhibitors is an MOR antagonist, an MOR partial antagonist, an MOR inverse agonist, an MOR partial inverse agonist, a KOR antagonist, a KOR partial antagonist, a KOR inverse agonist, a KOR partial inverse agonist, a DOR antagonist, a DOR partial antagonist, a DOR inverse agonist, a DOR partial inverse agonist, or a combination thereof.

5. The method of any of claims 1-4, wherein one or more opioid receptor inhibitors is one or more of alvimopan, AT-076 ((3R)-7-hydroxy-N-[(2S)-1-[4-(3-hydroxyphenyl)piperidin-1-yl]-3-methylbutan-2-yl]-1,2,3,4-tetrahydroisoquinoline-3-carboxamide), axelopran, bevenopran, buprenorphine, buprenorphine/samidorphan, buprenorphine/naltrexone, butorphanol, CERC-501 (4-(4-{[(2S)-2-(3,5-Dimethylphenyl)-1-pyrrolidinyl[methyl}phenoxy)-3-fluorobenzamide; CAS number 1174130-61-0), cyprodime, dezocine, diprenorphine, eptazocine, J-113,397 (1-R3R,4R)-1-cyclooctylmethyl-3-hydroxymethyl-4-piperidyl]-3-ethyl-1, 3-dihydro-2H-benzimidazol-2-one; CAS number 256640-45-6) or a racemic mixture thereof, JDTic ((3R)-7-Hydroxy-N-R2S)-1-[(3R,4R)-4-(3-hydroxyphenyl)-3,4-dimethylpiperidin-1-yl]-3-methylbutan-2-yl]-1,2,3,4-tetrahydroisoquinoline-3-carboxamide; CAS number 361444-66-8), JTC-801 (N-(4-amino-2-methylquinolin-6-yl)-2-[(4-ethylphenoxy)methyl]benzamide; CAS number 244218-51-7), levallorphan, levorphanol, LY-2940094 (2-[4-[(2-chloro-4,4-difluoro-spiro[5H-thieno[2,3-c]pyran-7,4′-piperidine]-1′-yl)methyl]-3-methyl-pyrazol-1-yl]-3-pyridyl]methanol; CAS number 1307245-86-8), methylnaltrexone, methylsamidorphan, nalbuphine, naldemedine, nalmefene, nalodeine, nalorphine, nalorphine dinicotinate, naloxegol, naloxone, 6β-naltrexol, naltrexone, naltrindole, norbinaltorphimine, pentazocine, PF-4455242 (2-Methyl-N-{[2′-(1-pyrrolidinylsulfonyl)-4-biphenylyl]methyl}-1-propanamine; CAS number 1202647-54-8), phenazocine, SB-612,111 (i.e., (5S,7S)-7-{[4-(2,6-dichlorophenyl)piperidin-1-yl]methyl}-1-methyl-6,7,8,9-tetrahydro-5H-benzol7lannulen-5-ol; CAS number 371980-98-2), samidorphan; or a salt, ester, or solvate of any of the aforementioned.

6. The method of any of claims 1-5, wherein one or more opioid receptor inhibitors is one or more of diprenorphine, levallorphan, nalmefene, nalorphine, nalorphine dinicotinate, naloxone, naltrexone, samidorphan; or a salt, ester, or solvate of any of the aforementioned.

7. The method of any of claims 1-6, wherein one or more opioid receptor inhibitors is one or more of diprenorphine, levallorphan, nalmefene, nalorphine, nalorphine dinicotinate, naloxone, naltrexone, or samidorphan.

8. The method of any of claims 1-7, wherein one or more opioid receptor inhibitors is nalmefene, naloxone, naltrexone, or samidorphan.

9. The method of any of claims 1-8, wherein one or more opioid receptor inhibitors is naltrexone.

10. The method of any of claims 1-9, wherein the amount of the one or more opioid receptor inhibitors is from about 0.0001% (by weight total composition) to about 99%.

11. The method of any of claims 1-10, wherein at least one of the one or more compositions further comprises a formulary ingredient.

12. The method of any of claims 1-11, wherein at least one of the one or more compositions is a pharmaceutical composition.

13. The method of any of claims 1-12, wherein at least one of the one or more administrations comprises parenteral administration, a mucosal administration, intravenous administration, depot injection, subcutaneous administration, topical administration, intradermal administration, oral administration, sublingual administration, intranasal administration, or intramuscular administration.

14. The method of any of claims 1-13, wherein at least one of the one or more administrations comprises a depot injection or an oral administration.

15. The method of any of claims 1-14, wherein if there is more than one administration at least one composition used for at least one administration is different from the composition of at least one other administration.

16. The method of any of claims 1-15, wherein the compound of at least one of the one or more compositions is administered to the animal in an amount of from about 0.005 mg/kg animal body weight to about 100 mg /kg animal body weight.

17. The method of any of claims 1-16, wherein the animal is a human, a rodent, or a primate.

18. The method of any of claims 1-17, wherein the animal is in need of treatment of fibrosis.

19. The method of any of claims 1-18, wherein the method is for treating lung fibrosis, skin fibrosis, kidney fibrosis, liver fibrosis, heart fibrosis, brain fibrosis, arterial stiffness, arthrofibrosis, crohn's disease, dupuytren's contracture, keloid, mediastinal fibrosis, myelofibrosis, peyronie's disease, nephrogenic systemic fibrosis, progressive massive fibrosis, a complication of coal workers' pneumoconiosis, retroperitoneal fibrosis, scleroderma/systemic sclerosis, or adhesive capsulitis.

20. The method of any of claims 1-19, wherein the method is for treating lung fibrosis, pulmonary fibrosis, cystic fibrosis, idiopathic pulmonary fibrosis (IPF), radiation-induced lung injury, skin fibrosis, kidney fibrosis, liver fibrosis, cirrhosis, heart fibrosis, atrial fibrosis, endomyocardial fibrosis, or myocardial infarction.

21. The method of any of claims 1-20, wherein the method is for treating lung fibrosis, pulmonary fibrosis, cystic fibrosis, idiopathic pulmonary fibrosis (IPF), radiation-induced lung injury, skin fibrosis, kidney fibrosis, heart fibrosis, atrial fibrosis, endomyocardial fibrosis, or myocardial infarction.

22. The method of any of claims 1-21, wherein the method is for treating lung fibrosis, kidney fibrosis, liver fibrosis, heart fibrosis, or brain fibrosis.

23. The method of any of claims 1-22, wherein the method is for treating lung fibrosis, kidney fibrosis, heart fibrosis, or brain fibrosis.

24. The method of any of claims 1-23, wherein the fibrosis is not liver fibrosis.

25. The method of any of claims 1-24, wherein the fibrosis is not fibrosis related to cirrhosis.

26. The method of any of claims 1-25, wherein the method further comprises one or more other fibrosis treatments.

27. The method of any of claims 1-26, wherein the method further comprises one or more other fibrosis treatments and the other fibrosis treatment comprises administering one or more of an antibiotic, an anti-inflammatory drug, a mucus thinner, or an antifibrotic medication.

28. The method of any of claims 1-27, wherein the method further comprises one or more other fibrosis treatments and the other fibrosis treatment comprises administering pirfenidone, nintedanib, or both.

29. The method of any of claims 1-28, wherein the method further comprises one or more other fibrosis treatments and the other fibrosis treatment comprises administering one or more non-drug respiratory therapies.

30. A method for treating a human for lung fibrosis, comprising one or more administrations of one or more compositions comprising naltrexone and optionally pirfenidone, nintedanib, or both, wherein the compositions may be the same or different if there is more than one administration.

31. A method for treating a human for IPF, comprising
administering one or more compositions comprising naltrexone and
optionally administering one or more compositions comprising pirfenidone, nintedanib, or both.
